(19) United States
(12) Patent Application Publication
(10) Pub. No.: US 2008/0167358 A1
(43) Pub. Date: Jul. 10, 2008

(54) Methods for Obtaining a Fast Dissolving Imidapril Powder

(76) Inventors: Marinette Moreau, Saint-Germain (FR); Sebastien Grellety, Ternuay (FR)

Correspondence Address: IP GROUP OF DLA PIPER US LLP, ONE LIBERTY PLACE, 1650 MARKET ST, SUITE 4900, PHILADELPHIA, PA, 19103, US

(21) Appl. No.: 11/885,806

(22) PCT Filed: Mar. 7, 2006
(86) PCT No.: PCT/FR06/00508

§ 371 Date: Nov. 6, 2007

(30) Foreign Application Priority Data
Mar. 7, 2005 (FR) 0502252

(51) Int. Cl. A61K 31/4164; A61K 9/16

(52) U.S. Cl. 514/392; 424/489

(57) ABSTRACT

A method for preparing a fast dissolving Imidapril powder including spraying a solution of Imidapril on at least one excipient in a granulator.

### RELATED APPLICATION

This is a §371 of International Application No. PCT/FR2006/000508, with an international filing date of Mar. 7, 2006 (WO 2006/095085 A1, published Sep. 14, 2006), which is based on French Patent Application No. 05/02252, filed Mar. 7, 2005.

### TECHNICAL FIELD

This disclosure relates to methods for preparing a fast dissolving Imidapril powder, particularly useful for reconstituting a solution intended for oral administration.

### BACKGROUND

Oral formulations are mainly solid formulations (powders, tablets, capsules) or liquid formulations (solutions, suspensions). Liquid forms are adapted, for example, in the case where the active substance is stable when in solution or in suspension.

In the case of unstable molecules, more particularly molecules which are unstable in an aqueous medium such as, for example, Imidapril, one way includes preparing a powder to be reconstituted in the medium, for instance an aqueous medium, prior to administration.

The rate of powder dissolution in the medium must then be as high as possible to facilitate preparation of the solution by the user. This dissolution rate depends on the temperature of the medium in which the powder is to be dissolved as well as the particle size and the crystalline condition of the powder.

This problem is all the more critical as it concerns Imidapril which is known to be a particularly unstable compound in an aqueous solution.

The techniques making it possible to prepare a fast dissolving powder have been widely developed. At present, the most commonly used techniques are co-precipitation, atomization, micronization, and freeze-drying techniques.

The article by G. F. Palmieri, I. Antonini and S. Martelli (Characterization and dissolution studies of PEG 4000/fenofibrate solid dispersion, S. T. P. PHARMA SCIENCES 6 (3) 188/199 1996) describes a method for obtaining solid dispersion by melting. A physical mixture composed of fenofibrate and polyethylene glycol 4000 is heated until the components are molten. The molten mixture is then cooled until it solidifies and the product thus obtained is crushed and sieved. In spite of its apparent simplicity, this method is difficult to operate on an industrial scale because of the sticky property of the solid block obtained.

M. Moneghini, A. Carcano, G. Zingone, B. Perissutti (Studies in dissolution enhancement of atenolol, Part I, International Journal of Pharmaceutics 175 (1998) 177/183) described a method for preparing solid dispersions based on the utilization of solvents. More particularly, the article describes a method for preparing a solid dispersion based on an active substance and polyvinylpyrrolidone, using an organic solvent. According to that method, the solvent is vacuum evaporated by raising the temperature. However, that method requires using and handling large volumes of organic solvents which represent primary disadvantages on an industrial scale.

The micronization of the active substance or the co-micronization with the water-soluble excipient are techniques well known to one skilled in the art to increase the dissolution rate of a substance. However, the micronized product is difficult to handle in the form of a powder. In this case, it is then necessary to perform additional operations such as granulation and drying to obtain an appropriate powder.

EP 0 973 506 describes a freeze-drying method making it possible to obtain a solid galenic form with a fast dissolving. That method is based on proportioning, in suitable containers, a solution or a suspension including the active substance, followed by freeze-drying. However, the manufacturing cost, brittleness of the obtained product and, more particularly, its friability as well as the need for an immediate container are important disadvantages for the implementation of such a method on an industrial scale.

The article by Özdemir and Ordu (Drug Development and Industrial Pharmacy, 24(1), 19/25, (1998)) describes a trituration method making it possible to enhance the dissolution properties of the Furosemide compound by chelation. A hydro-alcoholic solution is added to a mixture of Furozenol and of β-Cyclodextrin until a homogenous creamy product is obtained. The mixture is then ground using a mortar and a pestle, and vacuum dried. The complex mixture thus formed however requires some more operations to reach its galenic form.

Although they give satisfactory results, all the known methods for preparing quick dissolution powders have numerous disadvantages such as industrial cost, technical complexity of the method steps and, more particularly, the high number of operations required for obtaining a formulation which can be administered.

Considering the above, it could be advantageous to develop a simple and quick method making it possible to obtain a fast dissolving Imidapril powder and which does not present the disadvantages of the methods described in the prior art.

### SUMMARY

We provide a method for preparing a fast dissolving Imidapril powder including spraying a solution of Imidapril on at least one excipient in a granulator.

We also provide an Imidapril powder obtained from the method.

### BRIEF DESCRIPTION OF THE DRAWINGS

Other advantages and characteristics of our methods will appear evident when reading examples mentioned hereinunder and the appended figures in which:

FIG. 1 shows a view taken with an electronic microscope (magnification 50) of raw Imidapril, i.e., prior to being incorporated in our methods;

FIG. 2 shows a view taken with an electronic microscope (magnification 50) of the Imidapril powder as obtained after applying our methods (Example 1).

### DETAILED DESCRIPTION

We developed a method which is advantageously economical, simple and quick, and makes it possible to use equipment known and widespread in the pharmaceutical industry. The method makes it possible to obtain an Imidapril powder which can be directly packaged and which has good stability when stored.

Thus, we surprisingly found that it is possible to obtain Imidapril in a particular physical condition and, more notably, a new crystalline condition, whatever the initial physical condition of the Imidapril used, by spraying a Imidapril solution onto an excipient in a granulator, for instance, a fluidized bed granulator, and by drying the resulting Imidapril powder.

Such crystalline condition is different from that of Imidapril in that:

a different outer crystal morphology, more particularly by its surface finish;

a lower melting temperature (−29° C.).

Such new crystalline condition makes it possible to obtain a powder having a high dissolution rate, at least comparable not to say increased with respect to the dissolution rates described in the prior art for the known formulations of Imidapril (refer to Example 4 hereunder).

Thus, we provide a method for preparing a fast dissolving Imidapril powder comprising at least the steps of spraying a solution of Imidapril onto at least one excipient in a granulator, preferably a fluidized air bed granulator or a fluidized inert gas bed granulator and drying the powder thus obtained.

The powder obtained after drying may have an Imidapril/water-soluble excipient ratio between 1/2 and 1/20, preferably between 1/8 and 1/10 by weight with respect to the total weight of the powder.

More particularly, we provide a method for preparing an Imidapril powder comprising the following steps:

a) preparation of a Imidapril solution;

b) heating the Imidapril solution;

c) mixing and heating at least one excipient in a granulator, preferably a fluidized air bed or inert gas fluidized bed granulator;

d) spraying, in the granulator, the Imidapril solution of step b) onto the excipient of step c);

e) drying the powder obtained in the previous step.

Imidapril can have any form compatible with the use in a fluidized air bed granulator or a fluidized inert gas bed granulator, particularly in the form of a solution.

The solution can have any nature compatible with the spraying in a fluidized air bed granulator or a fluidized inert gas bed granulator such as, for example, an alcoholic, hydro-alcoholic or aqueous solution.

Advantageously, the Imidapril solution is an aqueous solution.

When the Imidapril solution is an aqueous solution, Imidapril may be contained in the aqueous solution in a concentration between about 1 and about 16%, preferably between about 10 and about 13% by weight with respect to the total weight of the solution.

The aqueous Imidapril solution can be heated up to a temperature between about 30° C. and about 70° C., preferably between about 55° C. and about 60° C.

Advantageously, the excipient can be heated up to a temperature between about 30° C. and about 70° C., preferably between about 50° C. and about 60° C.

Advantageously, the granulator can be an apparatus in which granulation and drying can be performed.

Selected devices are marketed under the names of Glatt® (manufactured by Glatt Agen, in Germany and Okawara Seisakusho Co., in Japan), Aeromatic® (manufactured by Aeromatic AG, in Switzerland and Fuji Industries Co., in Japan), Calmic® (manufactured by Calmic Engineering Co., in Great Britain), Growmax® (manufactured by Fuji Powdal Co., in Japan) and Flowcoater® (manufactured by Freund Industries Co., in Japan).

Spraying the Imidapril solution can be made at a rate which will depend on the equipment used and the manufacturer's specifications.

Advantageously, the inlet temperature of the air or inert gas flow can be between about 35° C. and about 90° C., preferably between about 60° C. and about 80° C.

Drying the obtained powder can be performed by any method known in the art. Advantageously, drying can be performed in the same device as granulation.

Drying the powder can be performed at a temperature between about 50° C. and about 90° C., preferably between about 70° C. and about 80° C.

The method may further include an additional step including calibrating the powder obtained after drying.

Calibration of the powder can be performed in any known device, particularly as an example, by an oscillating device marketed under the name of Erweka® fitted with a grid.

The excipient may be selected among excipients for pharmaceutical use.

Preferably, the excipient is an inert excipient.

In applications for aqueous solutions, the excipient is preferably a water-soluble excipient, more preferably an inert water-soluble excipient which may be selected among dextrin, dextrose or monohydrate glucose, erythritol, fructose, lactitol, lactose, maltitol, maltose, maltodextrin, mannitol, povidone, polyoxyethylene-glycols, sucrose or saccharose, sorbitol and xylitol, or mixtures thereof.

Preferably, the excipient maybe selected among lactose, maltodextrin, mannitol and povidone or mixtures thereof.

It is possible to use at least one additional excipient which can advantageously be chosen among preservatives, anti-oxidants, anti-microbials, flavors of synthetic or natural origin, pH modifiers and dilutants.

The additional excipient can be mixed with the Imidapril solution, with the water-soluble excipient or with the powder obtained by the method.

Advantageously, the mixture of the additional excipient and Imidapril can be made prior to or during the spraying operation.

Our methods make it possible to obtain a powder such that about 75 mg to about 300 mg of active substance contained in the powder are dissolved in about 30 ml of water at room temperature within a time at least lower than about 2 minutes, preferably lower than about 1 minute.

The powder obtained by our methods can be directly used either after the drying step or after the calibration step, more particularly to be packaged for example in vials and ampoules more particularly made of glass, tubes or bags or in the form of gels or tablets.

### EXAMPLE 1

### Obtaining a Powder Based on Imidapril, Lactose and Maltodextrin

A powder based on Imidapril, Lactose and Maltodextrin having the following final proportions by weight:

| | |
|---|---|
| Imidapril | 10.3% |
| Lactose | 77.3% |
| Maltodextrin | 12.4% |

was prepared according to the following protocol:

An aqueous starting solution comprising 13% by weight of Imidapril was heated to 60° C.

Lactose (Pharmatose DCL11) and Maltodextrin (Lycatab DSH) were loaded and mixed in a fluidized air bed device UniGlatt® fitted with a “top-spray” spraying nozzle. The mixture of excipients was then heated to 60° C.

The starting Solution of Imidapril was then sprayed in the granulator at a temperature of 60° C. at a rate of 9 g/min.

The air inlet temperature in the granulator was 70° C.

The temperature in the fluidization bowl was kept higher than 30° C. during the whole spraying step.

Drying the powder obtained after the spraying step was carried out in the granulator with an inlet air temperature of 70° C.

The drying step was then continued until a powder having a desiccation loss lower than 2% was obtained.

The characteristics of the powder based on Imidapril, Lactose and Maltodextrin obtained in Example 1 are described in Table I hereinafter.

TABLE I

| Characteristics | Results | |
|---|---|---|
| Appearance | Homogenous white powder | |
| Desiccation loss | 1.7% | |
| Apparent density | 0.43 | g/ml |
| Density after compaction | 0.46 | g/ml |
| Compaction capacity | 8 | ml |
| Flow | 14 | sec |
| Dissolution time for a quantity of 75 mg of | 30 | sec |
| the Imidapril powder obtained in Example | | |
| 1 in 30 ml of water at room temperature | | |

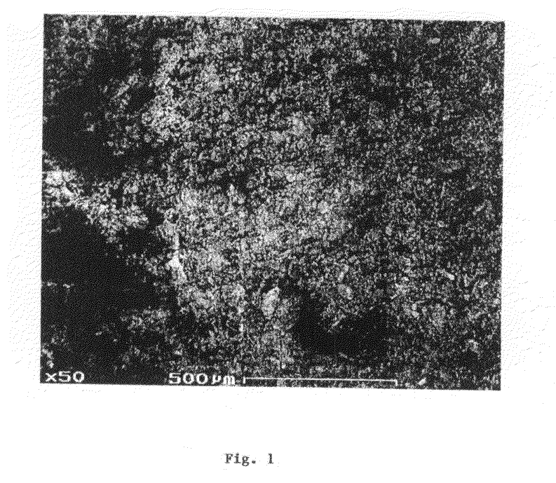

Surprisingly, a new crystalline condition of the Imidapril obtained by this method is observed as shown in FIG. 1.

This crystalline condition makes it possible to obtain a quick dissolution Imidapril powder, i.e., having a total dissolution time of the powder of 30 sec for a quantity of 75 mg of Imidapril in 30 ml of water at room temperature (25° C.).

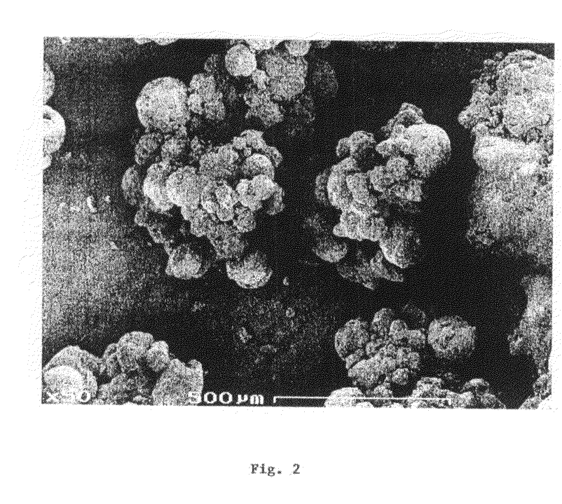

The thermal analysis showed the capacity of changing the condition of the Imidapril powder with respect to the initial condition of Imidapril. As a matter of fact, as shown in FIG. 2, the difference in melting temperature of the initial Imidapril and the powder amounts to +29° C.

### EXAMPLE 2

### Obtaining a Powder Based on Imidapril, Lactose and Povidone

A powder based on Imidapril, Lactose (Pharmatose DCL 11) and Povidone (Kollidon 30) having the following final proportions by weight:

| | |
|---|---|
| Imidapril | 10.3% |
| Lactose | 79.7% |
| Povidone | 10.0% |

as prepared according to the protocol of Example 1.

The characteristics of this powder based on Imidapril, Lactose and Povidone are described in Table II hereinafter.

TABLE II

| Characteristics | Results | |
|---|---|---|
| Appearance | Homogenous white powder | |
| Desiccation loss | 1.3% | |
| Apparent density | 0.53 | g/ml |
| Density after compaction | 0.58 | g/ml |
| Compaction capacity | 4 | ml |
| Flow | 9 | sec |
| Dissolution time for a quantity of 75 mg of | 15 | sec |
| the Imidapril powder obtained in Example | | |
| 2 in 30 ml of water at room temperature | | |

Surprisingly, a new crystalline condition of the Imidapril obtained by this method is observed, which corresponds to the crystalline condition obtained in Example 1.

### EXAMPLE 3

### Obtention of a Powder Based on Imidapril and Mannitol

A powder based on Imidapril and Mannitol having the following final proportions by weight:

| | |
|---|---|
| Imidapril | 10.3% |
| Mannitol | 87.9% |

was prepared according to the protocols of Example 1.

The characteristics of this powder based on Imidapril and Mannitol is described in Table III hereinafter.

TABLE III

| Characteristics | Results | |
|---|---|---|
| Appearance | Homogenous white powder | |
| Desiccation loss | 0.3% | |
| Powder density | 0.58 | g/ml |
| Powder density after compaction | 0.71 | g/ml |
| Compaction capacity | 12 | ml |
| Flow | 12 | sec |
| Dissolution time for a quantity of 75 mg of | 30 | sec |
| the Imidapril powder obtained in Example | | |
| 3 in 30 ml of water at room temperature | | |

Surprisingly, a new crystalline condition of the Imidapril obtained by this method is observed which corresponds to the crystalline condition obtained in Example 1.

### EXAMPLE 4

### Comparison of the Dissolution Rates of the Active Substance (Imidapril) Contained in Various Galenic Forms

Four formulations were made using the following compositions and procedures to compare the powder obtained by our methods to those obtained by the methods known in the art:

### Method No. 1: Co-precipitation

Formula:

| Components | Proportions (%) |
|---|---|
| Imidapril | 9.7 |
| Mannitol | 90.3 |
| Total | 100.0 |

Preparation method:

1. Preparation of an aqueous solution of Imidapril and Mannitol.

2. Vacuum evaporation of water.

3. Collection of the precipitate obtained.

### Method No. 2: Atomization

Formula:

| Components | Proportions (%) |
|---|---|
| Imidapril | 30.0 |
| Maltodextrin | 70.0 |
| Total | 100.0 |

Preparation method:

1. Dissolution of Imidapril in a hydro-alcoholic mixture heated up to 50° C.

2. Dissolution of Maltodextrin in water at 50° C.

3. Mixture of both solutions at 50° C.

4. Atomization of the solution.

5. Collection of the atomization results.

### Method No. 3: Micronization

Micronization of Imidapril was performed using an air jet microniser.

The granulometric characteristics are as follows, it being understood that “vd” means equivalent volume diameter, i.e., “the diameter of a sphere having the same volume as the particle.” As a matter of fact, as the forms of the particles vary, they are assimilated to fixed volume spheres so as to be compared.

The following table gives the average vd value in micrometer as well as the size distribution in the powder sample.

Thus, the average vd value with respect to the total sample amounts to 4.85 μm, the sample in which 10% of the particles have a diameter lower than 1.23 micrometers, 25% of particles have a diameter above 2.14 micrometers, 50% of the particles have a diameter lower than 3.75 micrometers, 75% of particles have a diameter lower than 6.10 micrometers and 90% of the particles have a diameter lower than 9.25 micrometers.

Average vd 4.85 μm

| Vd < | vd < | vd < | vd < | vd < |
|---|---|---|---|---|
| 1.23 μm | 2.14 nm | 3.75 μm | 6.10 μm | 9.25 μm |
| 10% | 25% | 50% | 75% | 90% |

### Method No. 4: Freeze-drying

Formula:

| Components | Proportions (%) |
|---|---|
| Imidapril | 9.32 |
| Mannitol | 86.96 |
| Sodium Benzoate | 3.72 |
| Total | 100.0 |

Preparation method:

1. Dissolution of sodium benzoate in water at 30° C.

2. Dissolution of Imidapril.

3. Dissolution of Mannitol.

4. Filtration of the obtained solution.

5. Distribution into suitable containers.

6. Freeze-drying.

Protocol for Comparing Formulations

A quantity of each formulation corresponding to 75 mg of Imidapril is added to 30 ml of water being stirred at room temperature. The apparent dissolution time is noted.

Results

The table hereinunder makes it possible to compare the dissolution times of ↓mg of Imidapril in water at room temperature for the various formulations.

| Formulation | Standard Imidapril | 1 | 2 | 3 | 4 | Invention |
|---|---|---|---|---|---|---|
| Time (s) | 420 | 60 | 35 | 26 | 20 | 30 |

The results obtained show that our methods make it possible to obtain a product of which the active substance dissolution rate is significantly enhanced with respect to that of the active substance in the form of raw material (Standard Imidapril). Besides, this dissolution rate is, at least comparable to the formulations known in the art, or even enhanced with respect to those.

1-18. (canceled)

19. A method for preparing a fast dissolving Imidapril powder comprising spraying a solution of Imidapril on at least one excipient in a granulator.

20. The method according to claim 19, wherein, in the powder obtained after drying, the Imidapril/excipient ratio is between 1/2 and 1/20 by weight with respect to the total weight of the powder.

21. The method according to claim 19, wherein, in the powder obtained after drying, the Imidapril/excipient ratio is between 1/8 and 1/1 by weight with respect to the total weight of the powder.

22. The method according to claim 19, further comprising:
a. preparing an Imidapril solution;
b. heating the Imidapril solution;
c. mixing and heating at least one excipient in a granulator;
d. spraying, in the granulator, the Imidapril solution of step b onto the excipient of step c; and
e. drying the powder obtained in step d.

23. The method according to claim 19, wherein the Imidapril solution is an alcoholic, hydro-alcoholic or aqueous solution.

24. The method according to claim 23, wherein the Imidapril is contained in the aqueous solution in a concentration between about 1 and about 16% by weight with respect to the total weight of the solution.

25. The method according to claim 23, wherein the Imidapril solution is heated to a temperature between about 30° and about 70° C.

26. The method according to claim 19, wherein the excipient is heated to a temperature between about 30° C. and about 70° C.

27. The method according to claim 19, wherein inlet temperature of air or inert gas flow is between about 35° C. and about 90° C.

28. The method according to claim 19, wherein drying the powder is obtained at a temperature between about 50° C. and about 90° C.

29. The method according to claim 19, further comprising a calibrating step of the powder obtained after drying.

30. The method according to claim 19, wherein the excipient is an inert excipient.

31. The method according to claim 19, wherein the excipient is a water-soluble excipient.

32. The method according to claim 31, wherein the excipient is at least one selected from the group consisting of dextrin, dextrose or mono-hydrated glucose, erythritol, fructose, lactitol, lactose, maltitol, maltose, maltodextrine, mannitol, povidone, polyoxyethylene glycols, sucrose or saccharose, sorbitol and xylitol.

33. The method according to claim 19, further comprising at least one additional excipient selected from the group consisting of preservatives and antioxidants, antimicrobials, flavors of synthetic or natural origin, pH modifiers and dilutants.

34. The method according to claim 33, wherein the additional excipient is mixed with the Imidapril solution, the water-soluble excipient or the powder.

35. The method according to claim 33, wherein the mixture of the additional excipient and Imidapril is made prior to or during spraying.

36. An Imidapril powder obtained from the method according to claim 19.
